(19) United States
(12) Patent Application Publication
(10) Pub. No.: US 2013/0330767 A1
(43) Pub. Date: Dec. 12, 2013

(54) Cell Lysis Process

(71) Applicants: John MacDonald Liddell, Billingham (GB); Azam Razzaq, Billingham (GB)

(72) Inventors: John MacDonald Liddell, Billingham (GB); Azam Razzaq, Billingham (GB)

(73) Assignee: FUJIFILM DIOSYNTH BIOTECHNOLOGIES UK LIMITED, Billingham (GB)

(21) Appl. No.: 13/983,168

(22) PCT Filed: Feb. 17, 2012
(86) PCT No.: PCT/GB2012/000164

§ 371 Date: Aug. 16, 2013

(30) Foreign Application Priority Data
Feb. 23, 2011 (GB) 1103138.2

(51) Int. Cl. C12N 1/06

(52) U.S. Cl. CPC C12N 1/06
USPC 435/69.1; 435/259; 435/91.4

(57) ABSTRACT

A process for cell lysis is provided. The process comprises passing a mixture comprising a suspension of cells and a lysis reagent through a flow-through reactor, wherein the mixture passes through the reactor with pulsed or superimposed oscillating flow. The process is preferably employed for the preparation of biological products, such as pDNA or inclusion bodies.

The present invention concerns a process for lysing cells, and in particular the extraction of plasmid DNA (pDNA) from cells.

Cell lysis is a commonly practiced method for the recovery of biological products from within cells. In many cases, the cells are contacted with a lysis reagent, commonly an alkaline solution comprising a detergent, or a solution of a lysis enzyme. The biological product can then be recovered from the lysis solution.

EP0811055 discloses the use of a static mixer to achieve cell lysis.

EP0967269 discloses the use of a fluidic vortex mixer for cell lysis.

According to the present invention, there is provided a process for cell lysis which comprises passing a mixture comprising a suspension of cells and a lysis reagent through a flow-through reactor, wherein the mixture passes through the reactor with pulsed or superimposed oscillating flow.

Any method of generating pulsed or superimposed oscillating flow in a flow through reactor known in the art can be employed in the process of the present invention. Methods of generating pulsed or superimposed oscillating flow include for example the use of alternating motion of some intrinsic elements of the reactor, such as the operation of reciprocating plate columns, where the oscillation is generated by means of motion of plates alternating between motion with and against the direction of net flow.

In other examples, oscillating flow is generated by the hydraulic transmission of a perturbation to the liquid contained in the reactor. This perturbation is typically generated by for example systems using positive displacement pumps (such as plug or membrane pumps) to introduce the feed into the reactor or by the use of pneumatic oscillating systems. In pneumatic oscillating systems, the oscillation is generated by means of a pressurised gas which propels the liquid contained in a parallel branch to the column,

In further examples, pulsed flow is generated by introducing liquid to a reactor through a pulsation chamber, which causes the pressure inside the chamber to increase. Once the pressure in the chamber is high enough, a valve means permits the egress of the liquid from the chamber into the reactor. The corresponding pressure drop in the pulsation chamber causes the valve to close.

In certain embodiments, a preferred method of generating oscillating flow comprises the use of an oscillating piston pump.

The process of the present invention can be employed to recover biological products, such as polynucleotides, for example pDNA, or proteins, such as periplasmic proteins or inclusion bodies from cells.

Biological products which can be recovered by the process of the present invention are commonly produced by the growth and harvesting of host cells, and preferably by microbial fermentation of recombinant microorganisms. The most preferred host cell is E. coil although many other types of cells can be employed. This includes other bacteria, for example Pseudomonads such as Pseudomonas fluorescens, yeast and higher eukaryotic cells. Examples include yeasts, such as Pichia pastoris, Saccharomyces cerevisiae and Kluyveromyces lactis, filamentous fungi such as Neurospora spp and the algae Chamydomomas.

Inclusion bodies which can be recovered by the process of the present invention are insoluble aggregates formed in the cytoplasm of bacterial cells such as E. coli, most commonly comprising protein and especially recombinant protein.

The process of the present invention is preferably employed for the extraction of pDNA. pDNA which can be extracted by the process of the present invention can be produced in one or more of multiple forms, such as supercoiled, linear and open-circular (i.e. nicked or relaxed) isoforms. The supercoiled pDNA isoform has a covalently closed circular form and the pDNA is negatively supercoiled in the host cell by the action of host enzyme systems. In the open-circular isoform, one strand of the pDNA duplex is broken at one or more places. For many plasmid applications, the supercoiled isoform is most preferred and is advantageously separated from the linear and open-circular isoforms. Plasmids for gene transfer, e.g. in-vitro DNA transformation or in-vivo gene therapy, may require a high percentage of the supercoiled plasmid isoform and a low percentage of open circular isoform. Therefore, the commercial need to obtain highly purified supercoiled plasmid DNA is extremely high. Methods to convert the open circular plasmid isoform to the supercoiled isoform are known in the art. For example, US20060057683 discloses a process where this is achieved enzymatically. Thus, in certain embodiments, following extraction using the present invention, pDNA in the open circular isoform is converted using methods established in the art to the supercoiled isoform.

Methods for the production of pDNA are well known in the art. pDNA may be natural or artificial, for example, cloning vectors carrying foreign DNA inserts. In many embodiments, the pDNA is in the size range of 1 kilobase to 50 kilobases. For example pDNA encoding expressed interfering RNA is typically in the size range of 3 kilobases to 4 kilobases.

Liquids comprising cells which can be employed in the process of the present invention include culture broths in which the cells have been grown. The culture broth may be employed without intermediate treatment, or may be treated to at least partially purify the culture medium, or to increase the concentration of cells for example by centrifugation. In many preferred embodiments, the liquid is a suspension of cells prepared by harvesting the cells from the culture broth, and then resuspending the cells, preferably in an aqueous buffer solution. Cells are harvested from the liquid by methods well known in the art, such as centrifugation or microfiltration.

When resuspension of cells is employed, the cells are preferably resuspended in an aqueous buffer, commonly with a pH in the range of from 4 to 10, and preferably at around neutral pH, for example from 7 to 9. The buffer salt concentration is commonly in the range of from 10-100 mM, such as in the range 20-80 mM. In certain embodiments, a particularly suitable buffer is 50 mM Tris HCl at pH 8. The buffer may contain chelating agents such as EDTA to maintain metal ions in solution and solubilise cell wall cations such as calcium. The resuspension buffer may also contain other compounds to assist in pDNA release such as polyols, for example sucrose, commonly in the range of from 2 to 15% w/w, preferably from 5 to 10% w/w; surfactants, for example Triton™ X-100 commonly in the range of from 1 to 5% w/w, preferably from 1 to 3% w/w; and/or chaotropes, for example urea, commonly at a concentration in the range of from 0.5 to 8M, preferably from 1 to 3M.

When the liquid comprising cells is a culture broth, the pH may be adjusted to a pH in the range of from 4 to 10, and preferably at around neutral pH, for example from 7 to 9. Chelating agents and other compounds to assist with release of cell contents, as described above for cell resuspension, may be employed if desired.

Lysis reagents which can be employed are well known in the art, and include aqueous alkaline detergent solutions and cell wall lytic enzymes, such as lysozyme. Preferred lysis reagents comprise alkaline detergent solutions, such as 0.1M aqueous sodium hydroxide containing 0.5% sodium dodecyl sulphate. The lysis reagent may comprise aqueous sugar solutions, such as sucrose solution and chelating agents such as EDTA, for example the well known STET buffer. In certain embodiments, the lysis reagent is prepared by mixing the cell suspension with an equal volume of lysis solution having twice the desired concentration (for example 0.2M sodium hydroxide, 1.0% sodium dodecyl sulphate).

After the desired extent of lysis has been achieved, the mixture comprising lysed cells is commonly contacted with a neutralising or quenching reagent to adjust the conditions such that the lysis reagent does not adversely affect the desired product. In many cases, the pH is adjusted to a pH of from 5 to 9 and preferably from 6 to 8, most preferably from 6.5 to 7.5 to minimise or prevent degradation of the cell contents. When the lysis reagent comprises an alkaline solution, the neutralising reagent preferably comprises an acidic buffer, for example an alkali metal acetate/acetic acid buffer. In many embodiments, lysis conditions, such as temperature and composition of the lysis reagent are chosen such that lysis is substantially completed whilst minimising degradation of the desired product. In many embodiments, lysis times of up to ten to twenty minutes, commonly about five minutes, are employed.

The mixture of cells and lysis reagent passes through a flow-through reactor with pulsed or superimposed oscillating flow, preferably the pulses or oscillations being substantially along the axis of net flow. In many embodiments, the pulsed or oscillating flow is at a frequency of up to 20 Hz, such as up to 15 Hz, preferably up to 10 Hz, and most preferably up to 5Hz. In certain embodiments, a frequency of 0.1 to 3 Hz, especially 0.5 to 2 Hz, is employed.

It will be recognised that the flow rate will be selected, along with the volume of the reactor, to achieve the desired residence time in the reactor to achieve the desired degree of lysis, and typically substantially complete lysis of the cells.

The amplitude and frequency of oscillation, baffle spacing, spacing between baffle and wall, and baffle design are selected to achieve the desired degree of mixing and to control shear forces. Shear forces are controlled to minimise or avoid damage to the desired product. Preferably conditions are controlled to achieve fluid strain rates of less than 1×10⁵ s⁻¹. The process according to the present invention permits simple control of both residence time and mixing efficiency to optimise product recovery and avoid damage to the product.

In many embodiments, the flow through reactor comprises a column which may be vertical, horizontal or inclined. Preferably vertical or inclined columns are employed, with the liquid entering the reactor towards or at the base of the column, and exiting the reactor at or towards the top. Most commonly, the column is cylindrical, and may comprise one or more baffles, preferably annular baffles. Two or more flow-through reactors may be connected in series if desired. The reactor may also comprise a heat exchanger, such as a jacket to allow the control of the reactor temperature, sample ports, flow meters, pH meters conductivity meters and similar process monitoring apparatus.

In many embodiments, the reactor is configured and operated to achieve an oscillatory Reynolds number (R_{osc}) of at least 10, preferably at least 50, and typically no more than 500, especially in the range of from 100 to 250. In certain embodiments, the reactor is configured and operated to achieve a net flow Reynolds number (R_{net flow}) of at least 5, and typically no more than 50, such as from 10 to 20. In certain preferred embodiments, the reactor is configured and operated to achieve a velocity ratio (R_{osc}: R_{net flow}) in the range of from 2 to 20, especially from 2 to 12.

In certain embodiments, the lysed cells exiting the flow-through reactor are collected in a vessel, preferably a stirred vessel, containing neutralising or quenching reagent. In other embodiments, the lysed cells are combined with neutralising or quenching solution in a further flow-through reactor, most preferably passing through this reactor with oscillating flow. In many preferred embodiments, the neutralising or quenching reactor comprises a baffled column. Use of a such a column in combination with superimposed oscillating or pulsed flow enables efficient mixing to be achieved so that neutralisation or quenching can be achieved rapidly at low shear, and hence minimising degradation of the desired product.

The process of the present invention is suited to the processing of products produced at small, medium or large scale. Small scale is typically regarded as a scale of up to 2 litres, commonly employing shake flasks. Medium scale is typically regarded as a scale of from 2 litres to 500 litres. Large scale is typically regarded as a scale of greater than 500 litres, such as up to 100,000 litres, for example from 1000 litres to 10,000 litres.

pDNA which has been extracted by the process of the present invention is commonly purified and isolated by methods known in the art. Examples of such methods include centrifugation, filtration, chromatography, diafiltration, precipitation such as addition of CTAB or as described in Lander et al U.S. Pat. No. 6,797,476 and two phase aqueous extraction as described by Hubbuch et al, Biotechnol Appl Biochem. (2005) 42 pp57-66.

Large cell debris, protein and most genomic DNA is commonly removed by centrifugation. An optional treatment with RNase may be employed, and the pDNA may be filtered to further remove small debris, for example filtration through a 0.45 micron filter,

Further impurities may be removed by diafiltration, commonly using an ultrafiltration membrane having a molecular weight cut off selected according to the size of the pDNA.

Chromatographic methods which can be employed include charged membrane chromatography (for example as described in Endres et al, Biotechnol Appl Biochem. (2003) 37 pp259-66), monolith chromatography (for example as described in Stancar et al, Adv Biochem Eng Biotechnol. (2002) 76: pp49-85), anion exchange chromatography HIC and reversed phase chromatography. In many embodiments, anion exchange or HIC and reversed phase methods are employed. It is preferred that at least one, and preferably each of centrifugation, filtration and diafiltration steps are employed prior to chromatography. Examples of suitable anion exchange matrices include those available from POROS Anion Exchange Resins, Qiagen, Toso Haas, Sterogene, Spherodex, Nucleopac, and GE Healthcare. Examples of suitable reversed phase matrices include those available from POROS, Polymer Labs, Toso Haas, GE Healthcare, PQ Corp., Zorbax, BioSepra resins, BioSepra Hyper D resins, BioSepra Q-Hyper D resins and Amicon. Preferably, anion exchange chromatography precedes reversed phase chromatography. Examples of suitable HIC resins include Sepahroses, eg phenyl, butyl and octyl Sepharose, and Toyopearls, eg Toyopearl hexyl, butyl and phenyl.

Purified pDNA may be concentrated and/or diafiltered to reduce the volume or to change the buffer, for instance to transfer the pDNA into a pharmaceutically acceptable carrier or buffer solution, optionally followed by sterilisation. Examples of pharmaceutically acceptable carriers or buffer solutions are known in the art. Methods suitable for concentrating pDNA are well known in the art and include diafiltration, alcohol precipitation and lyophilisation, with diafiltration being preferred. Methods of sterilisation which do not affect the utility of the pDNA are well known in the art, such as sterilisation by passage through a membrane having a small pore size, for example 0.2 microns and smaller.

Inclusion bodies extracted by the process of the present invention are commonly purified by methods known in the art. Proteinaceous inclusion bodies are commonly solubilised and then refolded to produce functional native protein which would then be purified by orthogonal chromatography chemistries such as ion exchange, hydrophobic interaction chromatography, hydrophobic charge induction chromatography, affinity chromatography, immobilised metal affinity chromatography, rp-hplc, and size exclusion chromatography. In many instances, the protein is then sterile filtered, for example, through a 0.2 micron filter.

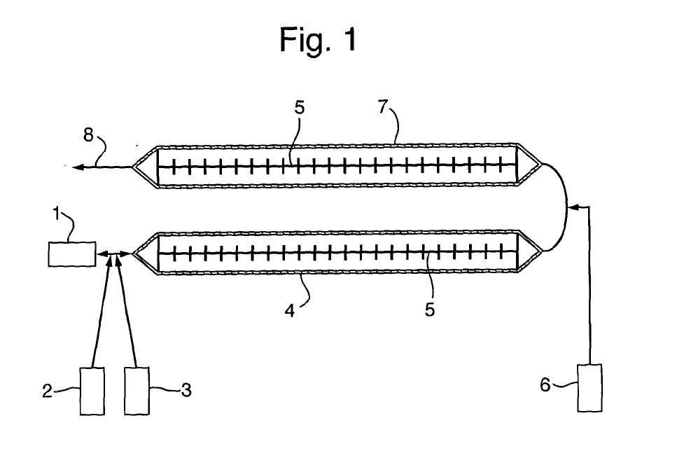

The process of the present invention is illustrated in FIGS. 1, 3 and 4. In FIG. 1, feed pumps 2 and 3 supply buffered cell suspension and NaOH/SDS solution, respectively, into a flow-through reactor, 4, which is fitted with a stringer, 5, running longitudinally through the reactor, and fitted with equally spaced baffles. Oscillating pump, 1, provides oscillating flow of the reaction medium through the reactor. On exiting reactor 4, the reaction medium is contacted with potassium acetate solution, supplied by pump, 6. The reaction mixture plus potassium acetate solution then flows through a second flow through reactor, 7, which is also fitted with a longitudinal stringer having equally spaced baffles, and then exits at outlet, 8.

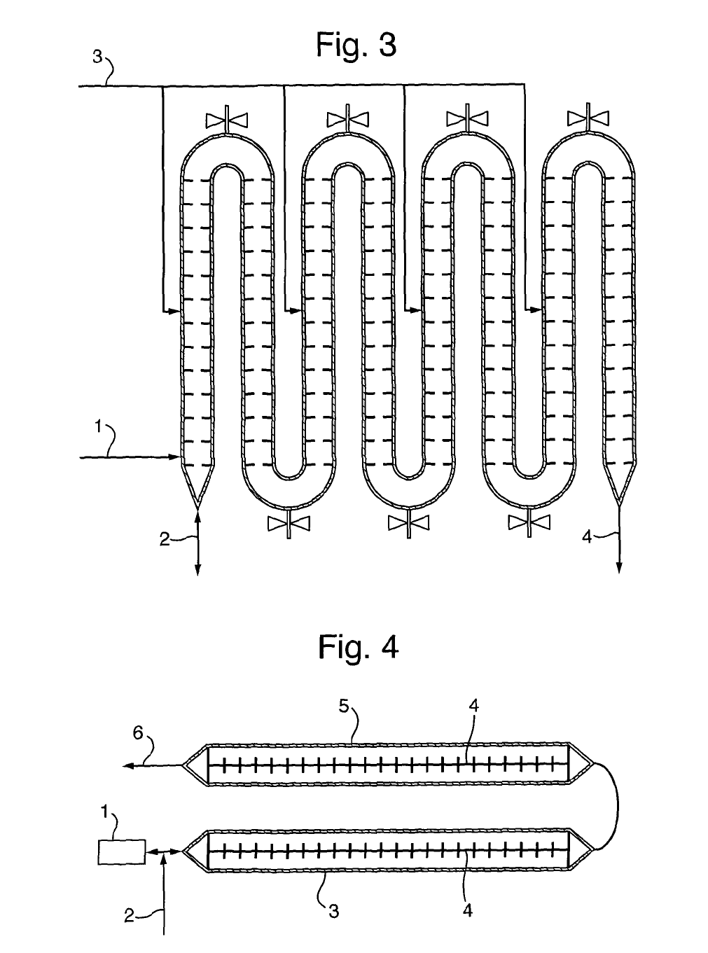

In the configuration shown in FIG. 3, cell suspension is pumped into a baffled reactor at 1. Oscillating flow is provided by an oscillator, 2. As the cell suspension flows through the reactor, desired reagents can be added through one or more reagent inlets, 3, and then exits the reactor at outlet, 4.

In the configuration shown in FIG. 4, cell suspension and lysis reagents are pumped into a baffled reactor at 2. Oscillating flow is provided by an oscillator, 1. The cell suspension and reagents flows through a plurality of flow-through reactors 3 and 5, each fitted with a longitudinal stringer having equally spaced baffles, 4, and then exits the reactor at outlet, 6.

The present invention is illustrated without limitation by the following examples.

### Strain Preparation

A recombinant E. coli XL1 Blue strain containing a plasmid encoding for the anti hen egg white lysozyme Fab D1.3 was used in the experimental studies. The size of the plasmid containing the D1.3 gene was ˜7.5 kb.

### EXAMPLES

40 μl of a glycerol stock of the E. coli strain was inoculated into 5 mL of LB (Luria broth) medium supplemented with 10 mg/mL tetracycline and this was then incubated at 37° C. overnight (˜16 h) with shaking at 200 rpm. 50 mL of this overnight culture was then inoculated into a 2 L baffled shake flask containing 0.5 L of LB media (supplemented with 10 mg/mL tetracycline) and grown overnight (˜16 h) at 37° C. with shaking at 200 rpm. The 0.5 L shake flask culture was then harvested by centrifugation at 16,000 g for 5 mins. The supernatant fraction was decanted to waste and the cell pellet was resuspended into a Cell Resuspension Buffer P1 (50 mM Tris, 10mM EDTA, pH 8.0), with a desired target of 5% (w/v) concentration. Resuspension of the 7.7 g wet cell pellet was achieved by vortexing the mixture of cell pellet in the presence of 170 mL Buffer P1, providing a 4.5% w/v homogenous cell suspension. This resulting 180 mL cell suspension was the starting feed material for the Continuous Oscillating Baffled Reactor (“COBR”) system, configured as illustrated in FIG. 1.

The COBR system consisted of two glass walled tubes arranged in series, thus generating two flow-through reactors with superimposed oscillating flow. Each tube was 7 mm in diameter, 46.5 cm in height, and contained 32 internal baffles, each 1 cm apart, thus generating baffled chamber volumes of 0.45 mL (18.5 mL volume per flow-through reactor). Three pumps (connected via a 3-way valve system) fed into the first flow-through reactor. The first pump fed the cell suspension into the reactor and the second pump fed the lysis solution, Buffer P2 (0.2M sodium hydroxide, 1% sodium dodecyl sulphate). The third pump (a Sapphire Engineering PVM metering pump fitted with a 5 ml syringe) provided the oscillation flow. A fourth pump fed a Neutralisation Buffer P3 (3M sodium acetate, 2M acetic acid, pH 5.5) into the second reaction chamber. The total volume of the COBR system was 37 mL, including the 3-way valve set-up, the two reaction chambers and connecting tubing.

Equal volumes of cell suspension and Buffer P2 were fed into the first oscillating reactor at the same feed rate. To control the lysis reaction time to 5 minutes, the flow rate into the first reactor was set at 1.85 mL/min for each feed, representing a combined feed rate of 3.7 mL/min, thus generating a residence time of 5 mins. As the cell lysate passed into the second flow-through chamber after the 5 min lysis time, it was neutralised by continuous addition of Buffer P3 (3M sodium acetate. As the feed rate of Buffer P3 was also 1.85 mL/min, the residence time of the neutralised lysate in the second reactor was 3.4 mins. The lysate was collected as a single bulk from the exit of the second flow-through chamber. During the reaction, Pump 3 provides the oscillation flow component, generating a 0.1 cm displacement at frequency of 3 Hertz. Spot samples from the outlet flow were taken at 90 mL intervals and were analysed for plasmid DNA content. Over the course of the COBR experiment, 180 mL of cell suspension was mixed with a total of 180 mL of Buffer P2 and 180 mL of Buffer P3, generating a 540 mL cell lysate over a period of 90 mins.

Plasmid DNA analysis of the COBR lysates was carried out by taking 0.6 mL samples of the final bulk and the 90 mL spot samples were clarified through a 0.45 μm filter. To assess the efficiency of recovery of plasmid DNA from the COBR experiment, a comparison was made to recovery from a positive control, which in this case was a standard laboratory mini-prep from 1.5 mL of the same culture used to generate cells for the COBR experiment. For the mini-prep, the cells were resuspended to a 4.4% cell suspension, equivalent to that of the COBR cell suspension. Buffers P1, P2 and P3 used for the COBR experiment were also used for the mini-prep. The COBR and mini-prep extracts were concentrated by DNA precipitation, using the addition of 0.8 volumes of 100% isopropanol. The subsequent clarified DNA pellets were washed with 70% ethanol, before resuspension into 504 of purified water.

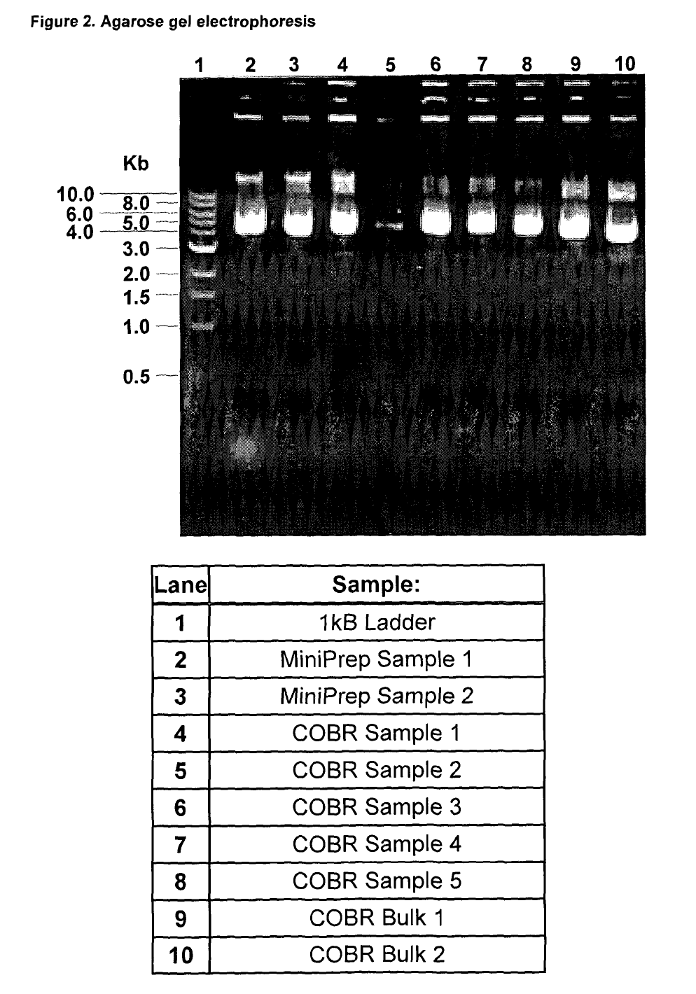

Agarose gel electrophoresis of two replicate mini-prep samples together with COBR samples 1-5 (reaction volume points 90 mL, 180 mL, 270 mL, 360 mL & end respectively), and 2 duplicate samples for the final bulk extract, (FIG. 2) showed that the level of plasmid DNA extraction in the COBR reactions is qualitatively equivalent to that from the minipreps. In addition, the level of pDNA generated over the course of the COBR experiment was equivalent to that in the final bulk sample.

Quantitative AIEX HPLC (anion exchange high performance liquid chromatography) showed that the average mini-prep yield from this experiment was 54.5 ±7.3 μg/mL of plasmid DNA, compared with the 53.6±0.2 μg/mL for the COBR bulk (Table 1). As the same amount of cell suspension was put into each reaction, these results show that the level of plasmid DNA extracted by each process was equivalent. Moreover, the average level of plasmid DNA recovery for the different COBR time point samples was 46.8±5.5 μg/mL, confirming that the level of extraction over the course of the COBR reaction was consistent and comparable to that of the bulk COBR sample.

TABLE 1
AIEX HPLC analysis of samples from COBR reaction. Two replicate
mini-prep samples, as well as COBR spot samples and two duplicate
COBR bulk samples were analysed and quantified against a plasmid
DNA standard curve.

| | DNA concentration | Average DNA concentration ± |
|---|---|---|
| Sample | (μg/mL) | standard deviation (μg/mL) |
| Mini-prep 1 | 49.3 | 54.5 ± 7.3 |
| Mini-prep 2 | 59.7 | |
| COBR 90 mL | 54.7 | 46.8 ± 5.5 |
| COBR 180 mL | 46.3 | |
| COBR 270 mL | 43.0 | |
| COBR 360 mL | 43.1 | |
| COBR Bulk 1 | 53.7 | 53.6 ± 0.2 |
| COBR Bulk 2 | 53.5 | |

1. A process for cell lysis which comprises passing a mixture comprising a suspension of cells and a lysis reagent through a flow-through reactor, wherein the mixture passes through the reactor with pulsed or superimposed oscillating flow.

2. A process according to claim 1, wherein the mixture passes through the reactor with superimposed oscillating flow.

3. A process according to claim 2, wherein the oscillating flow is at a frequency of up to 20 Hz.

4. A process according to claim 1 which is operated with fluid strain rates of less than 1×10⁵ s⁻¹.

5. A process according to claim 1, wherein two or more flow-through reactors are employed in series.

6. A process according to claim 5, wherein a quenching reagent is added to the mixture after it has passed through the first flow-through reactor.

7. A process for producing a biological product, comprising the steps of:
a) culturing host cells producing the biological product;
b) lysing the cells by a process according to claim 1; and
c) recovering the biological product.

8. A process according to claim 7, wherein the biological product is pDNA or an inclusion body.

9. A process according to either of claim 6, wherein the host cell is E. coli.

10. A process according to claim 1 or claim 7, wherein the reactor is configured and operated to achieve an oscillatory Reynolds number of at least 10, and no more than 500.

11. A process according to claim 1 or claim 7, wherein the reactor is configured and operated to achieve a net flow Reynolds number of from 5 to 50.

12. A process according to claim 1 or claim 7, wherein the reactor is configured and operated to achieve a velocity ratio in the range of from 2 to 20.

13. A process according to claim 7, wherein the mixture passes through the reactor with superimposed oscillating flow at a frequency of 0.1 to 3 Hz and which is operated with fluid strain rates of less than 1×10⁵ s⁻¹ and the reactor is configured and operated to achieve an oscillatory Reynolds number in the range of from 100 to 250.

14. A process according to claim 13, wherein the reactor is configured and operated to achieve a net flow Reynolds number of from 10 to 20 and to achieve a velocity ratio in the range of from 2 to 12.
